(19) United States
(12) Patent Application Publication
(10) Pub. No.: US 2012/0164722 A1
(43) Pub. Date: Jun. 28, 2012

(54) LIGHTING APPARATUS FOR A BIOREACTOR TO ENHANCE BIOLOGICAL MATERIAL GROWTH

(76) Inventor: Ian Malcolm Wright, Stapylton (AU)

(21) Appl. No.: 13/381,040

(22) PCT Filed: Feb. 19, 2010
(86) PCT No.: PCT/AU2010/000193

§ 371 Date: Mar. 15, 2012

(30) Foreign Application Priority Data
Feb. 20, 2009 (AU) 2009900738

(51) Int. Cl. C12M 3/00; C12M 1/00; F21V 5/00; F21V 5/04; F21V 21/00; F21V 21/14

(52) U.S. Cl. 435/292.1; 362/249.02; 362/249.03; 362/235

(57) ABSTRACT

A lighting apparatus (10) for a bioreactor including a plurality of coloured light emitting diodes (11) mounted on a support (21), the number and colour of the light emitting diodes chosen to provide predetermined spectral wavelengths and an optical device (13) to transmit the emitted light to a biological material to enhance the growth rates of said biological material including plants, flora and some species of bacteria and algae; wherein the support (21) is further mounted on two plates (17, 18) on support legs (15) serving as terminals to provide electrical power to the light emitting diodes. A bioreactor (not illustrated) is also disclosed which includes a rigid core, an outer growth containment portion, a lower end portion and an upper end cap, wherein the outer containment portion expands as a cellular biological material contained in said portion grows and expands.

### FIELD OF THE INVENTION

The present invention relates to lighting apparatus and more particularly to a lighting apparatus and system for providing lighting to a bioreactor.

### BACKGROUND ART

Conventional lighting apparatus are known and there are a wide variety of both lighting apparatus and applications to which they can be put.

It is an emerging field of study in the field of botanics that the growth rates of plants and other flora and indeed some species of bacteria and algae may be affected through the application of light of a particular wavelength. One substantial problem existing in the field is the application of the particular wavelengths to the growing material.

At the same time, inventors are continually looking for methods of increasing efficiency of growth whilst reducing the costs of producing the growing material.

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to a lighting apparatus, which may at least partially overcome at least one of the abovementioned disadvantages or provide the consumer with a useful or commercial choice.

With the foregoing in view, the present invention in one form, resides broadly in a lighting apparatus including a plurality of coloured light emitting diodes mounted on a support, the light emitting diodes chosen to provide predetermined spectral wavelengths and an optical device to transmit the emitted light.

According to an alternative embodiment, the present invention resides broadly in a bioprocess lighting system for providing light to a biological material to enhance growth, the lighting system including a plurality of light emitting diodes mounted on a support, the light emitting diodes chosen to provide optimum spectral wavelengths to the biological material and an optical device to transmit the emitted light for illumination of the biological material.

According to a further embodiment, the present invention resides in a bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands, the light source including a plurality of light emitting diodes mounted on a support, the light emitting diodes chosen to provide optimum spectral wavelengths to the culture medium and an optical device to transmit the emitted light for illumination of the culture medium.

The inventor has found that effective lighting provided to growing biological material, in particular plant material and other biologically cultured material and can result in an increase of the growth of the material (both growth rate and amount). This is particularly the case in a bioreactor designed to culture the biological material.

The ability to produce a predetermined spectral wavelength or wavelength band has enormous application in a wide variety of fields from treatment of disease to providing lights without the heating end of the visible spectrum and other applications.

The inventor has found that he can reduce the power consumption whilst creating an equivalent amount of light by using the lighting system of the present invention up to 1/80th of the conventional consumption. Light density, measured in lux is also important to cultivation in a bioreactor. The apparatus and system of the present invention produces high light density at lower power consumption.

As intimated above, the lighting apparatus and system of the present invention finds particular application with the use of bioreactors. The apparatus and system of the present invention is well adapted for use and particularly preferred for use with a bioreactor which is substantially cylindrical and which stands about a vertical axis. Such a bioreactor will normally have a rigid core associated with a light source, and an outer expandable growth containment portion located concentrically about the rigid core in which the biological material to be grown resides.

However, it is also anticipated that the lighting apparatus of the present invention may find application in the area of ultraviolet lighting apparatus (such as are used in ultraviolet sterilisers or ultraviolet lamps), infrared lighting apparatus, operating lamps with particular light spectrum to highlight or contrast blood and tissue or other application where the provision of light having a particular wavelength or range of wavelengths could be advantageous.

For the purpose of the present discussion, the lighting apparatus is discussed in one preferred application, namely for providing light to a bioreactor.

The lighting apparatus of the present invention includes a support which according to the most preferred embodiment used on the cylindrical bioreactor, is typically circular. Normally, the support of the present invention will be a part of or form a bottom or top plate for the bioreactor.

The lighting apparatus of the present invention is normally a powered electrical apparatus and as such, requires electrical connections to a power source. The electrical connections normally extend at least into the support from below (or above) as separation from the biological material which is to be cultured in the bio reactor is preferred as the biological material is normally at least partially liquid.

The preferred method of providing electrical power to the light emitting diodes is through the provision of power supply support “legs”. Typically, each leg will be a positive or negative terminal connected in turn to the power source. Typically, the support legs will be extend perpendicularly to the plane of the support and extend at least partially into the support.

According to a particularly preferred embodiment, an inner terminus of the support legs (defined relative to the support and being on the side of the support with the LED's) will preferably stand slightly proud of an upper surface of the support. The respective inner termini may be used as positive location means for a cover or the optical device.

Preferably, there will be a number of positive terminal legs and a lesser number of negative terminal legs. For the most preferred embodiment, for terminal legs will be provided and three of these will be positive terminal legs with only one being a negative terminal leg. All of the terminal legs will have appropriate connections to the light emitting diodes. The connection configuration is further discussed below.

The support of the present invention will typically be provided in one of two preferred implements namely, as a substantially planar plate or as a tiered system. When provided as a substantially planar plate, the support will normally form the bottom or top plate for the preferred cylindrical bioreactor.

When provided as a tiered system, there will typically be an outer plate and a spaced inner plate. Preferably, the outer plate is of larger dimension than the inner plate. Normally, the inner plate will be maintained above the outer plate by a shoulder wall which will preferably be continuous in order to define an internal space between an inner surface of the outer plate, the outer surface of the inner plate and the inner surface of the shoulder wall.

Typically, the electrical connection system between the terminal legs and the light emitting diodes will be located either in the space between the respective plates or alternatively, below the outer plate.

Normally, the light emitting diodes will be mounted on an appropriate mounting plate which is itself then subsequently mounted on the support. Preferably, the electrical connections with the light emitting diodes will extend through the support to connect with an outer side of the mounting plate.

According to a preferred embodiment, the mounting plate will be a printed circuit board containing electrical connections for the light emitting diodes. Normally, the mounting plate will allow replacement of the light emitting diodes. The mounting plate will normally be substantially centrally mounted relative to the support and will maintain the light emitting diodes facing inwardly toward the bioreactor.

Normally, the inner plate is smaller diameter than the outer plate. The provision of the larger dimension outer plate and a shoulder wall provides a section of the support, namely the shoulder wall and the inner plate, which may be used to positively locate the support with in the bioreactor. For example, normally a terminal end of the outer expandable growth containment portion can be located about the shoulder wall and inner plate and abutting the outer plate. A circumferential clamp means may then be used to clamp or otherwise secure the outer expandable growth containment portion relative to the shoulder wall.

It is also anticipated that the lighting system of the present invention may be provided as a unit separately from the apparatus in relation to which it is used. As a preferred embodiment is particularly adapted to use with a bioreactor, the lighting system may not be attached to the bioreactor. In this regard, the light emitted by the light emitting diodes may be transmitted in any appropriate manner to the apparatus in relation to which it is used. Typically, the optical device used to transmit the emitted light may include an elongate fibre-optic cable for example, in order to transmit the emitted light to the location of its use.

The apparatus and system of the invention includes a plurality of light emitting diodes (LEDs). Any number of LEDs may be provided but, a square number is preferred for ease of electrical connection.

As stated above, the number and combination of colours of the LEDs provided will be selected to provide an optimum wavelength of emitted light. Normally, the optimum wavelength will be within a particular range of wavelengths and the range will be chosen according to the biological material or plant material to be cultured.

For example, even though photosynthesis (and thus growth) is almost equally efficient across the entire visible spectrum from 400 to 700 nm, chlorophyll absorbs maximally in the blue and red portions of the spectrum.

As a further example, LED's emitting a lot of short wavelengths (380-470 nm) have been found to induce astaxanthin accumulation of up to 5 to 6% per dry cell, although the induction course the suppression of cell growth in the photosynthetic microalgae Haematococcus pluvialis, a potential source of astaxanthin. Therefore, the growth of this microalgae under the elimination of red LEDs (λmax=625 nm) to allow for cell growth without inducing a higher level of astaxanthin accumulation, and in switching to elimination with blue LEDs (λmax=470 nm) at a high light intensity to induce a high level of astaxanthin has been used effectively.

It is also a particularly preferred feature of the present invention that the cluster of LEDs are adjustable in order to adjust the available applied wavelengths of light during the various growth phases of the biological medium as required to stimulate or encourage growth during that particular growth phase.

Normally, the component LEDs used in the cluster for a particular application will be chosen in order to provide a range of wavelengths and normally, the individual LEDs will be actuable to allow adjustment of the wavelengths emitted from a particular cluster. For example, an LED cluster of the present invention may include LEDs of each of the component colours, for example, red (λmax=625 nm), green (λmax=525 nm), blue (λmax=470 nm), blue-purple (λmax=410 nm), purple (λmax=380 nm), ultraviolet ((λmax=less than 380 nm) and infrared (λmax=greater than 625 nm). By having particular LEDs in the cluster in the light and others not emit light, a wavelength range of any desired wavelength can be emitted from the apparatus. Not only that, but by switching the particular combinations which are lit and unlit, the wavelength range can be adjusted during the growth phases of the material.

As outlined above, the plurality of LEDs are normally mounted on a printed circuit board in an array, the array is normally a regular array and according to a particularly preferred embodiment, includes nine or 16 LEDs.

Typically, the LEDs are connected in the most efficient method electrically. Normally, the negative terminal will be connected to each of the rows of LEDs according to a pattern similar to the shape of the Greek letter psi (Ψ). Other configurations may be used.

The apparatus of the present invention also includes an optical device to transmit the emitted light. In its simplest form applied to use in the bioreactor field, the optical device may be a transparent or translucent cover placed over the LED cluster in order to emit the light directly into the bioreactor.

Other more advanced optical devices may be used for particular applications. For example, the optical device may be a lens which is adapted to focus or disperse the emitted light. The optical device may therefore be a convex or concave shape depending upon requirements.

Alternatively, the light emitted by the LED cluster may be conveyed, transmitted or spread using a fibre-optic cable. In order to be used in this manner, the emitted light may need to be focused to a smaller cross-section beam, and in this instance, a focusing lens may be used.

More than one lighting apparatus according to the present invention may be used, particularly in the bioreactor field where as the biological material grows, it will usually absorb the light and prevent the light from reaching the more central portions of the biological mass. In this instance, it may be beneficial to agitate the biological mass in the bioreactor in order to expose all areas to the emitted light or alternatively, provide enough lighting apparatus spaced around the bioreactor to expose all portions of the biological mass to the emitted light.

In a further alternative form, the emitted light may be dispersed. One mechanism for doing this is providing a shaped reflector in order to disperse the light. A further example of a mechanism for dispersing the light is to score the outside of a fibre-optic cable used to transmit the light, in channels in order to allow the light to escape from the fibre-optic cable.

Other alternatives are possible such as light scattering additives or reflective additives. The additives may be added at the macro or microlevel.

According to a further embodiment, the present invention resides in a bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands radially under the pressure exerted by the growth of the biological material.

It is preferred that the LED's used are individually filtered.

### BRIEF DESCRIPTION OF THE DRAWINGS

Various embodiments of the invention will be described with reference to the following drawings, in which:

FIG. 1 is a schematic view from above of a lighting apparatus according to a preferred embodiment of the present invention.

FIG. 2 is a schematic view from the side of the lighting apparatus illustrated in FIG. 1.

FIG. 3 is a perspective view of a support with mounting plate and terminal legs according to a preferred embodiment of the present invention.

FIG. 4 is a perspective view of a mounting plate, terminal legs and electrical connections according to a preferred embodiment of the present invention.

FIG. 5 is a perspective view from below of the configuration illustrated in FIG. 4.

FIG. 6 is a partially sectional perspective view of the configuration illustrated in FIG. 3, along line A-A.

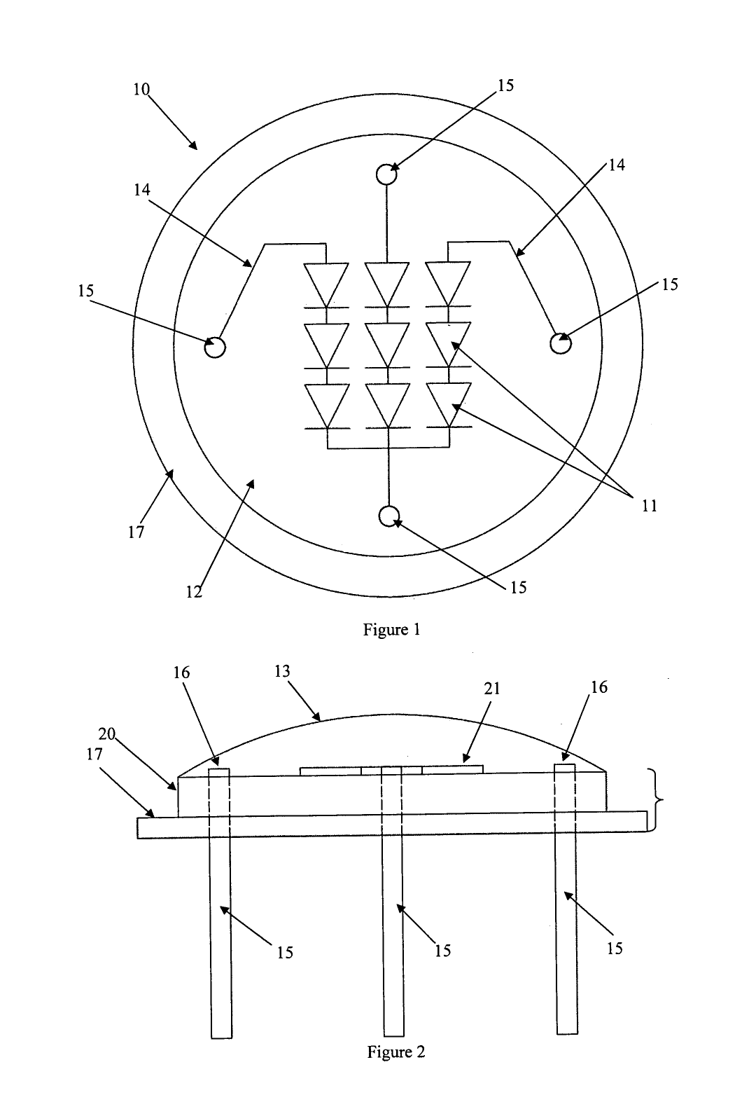

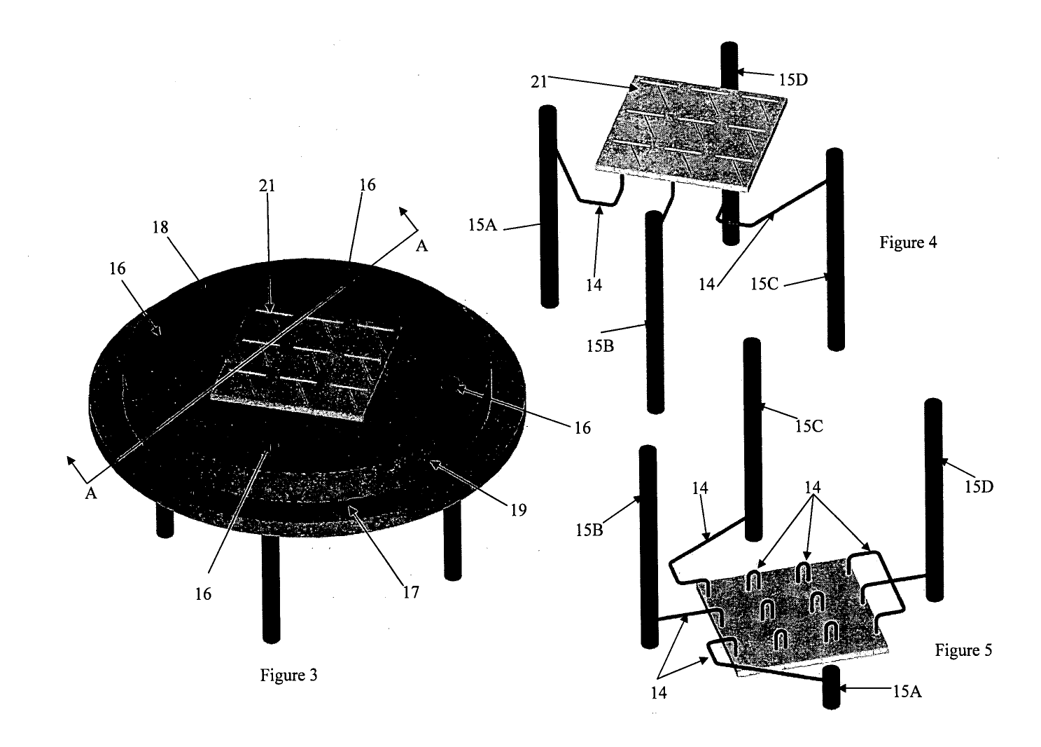

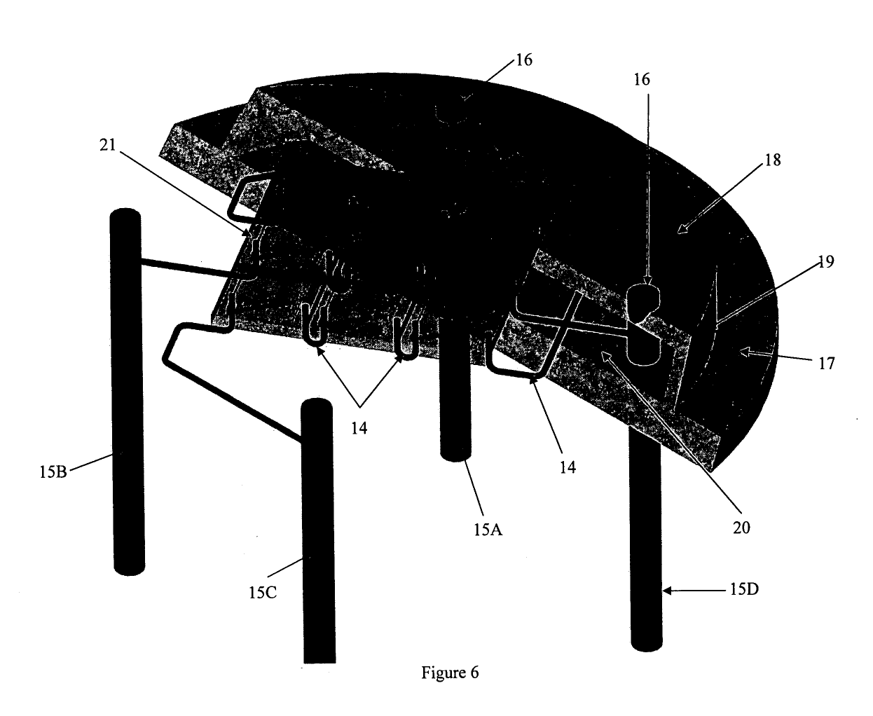

### DETAILED DESCRIPTION OF THE PREFERRED EMBODIMENT

According to a particularly preferred embodiment of the present invention, a lighting system for a cylindrical bioreactor (not illustrated) is provided.

As illustrated in the Figures, the lighting system 10 includes a plurality of coloured light emitting diodes 11 mounted on a support 12, and an optical device to 13 transmit the emitted light. The number and colour of the light emitting diodes 11 are chosen to provide predetermined optimal spectral wavelengths and to provide the ability to adjust the wavelengths without removing the lighting system from the cylindrical bioreactor.

The inventor has found that effective lighting provided to growing biological material, in particular plant material and other biologically cultured material and can result in an increase of the growth of the material (both growth rate and amount). This is particularly the case in a bioreactor designed to culture the biological material.

The lighting system 10 of the present invention includes a support 12 which according to the illustrated embodiment used on a cylindrical bioreactor (not illustrated), is circular.

The lighting system of the present invention is normally a powered electrical apparatus and as such, requires electrical connections to a power source. The electrical connections or wires 14 of the illustrated embodiment extend into the support 12 from below (or above) as separation from the biological material which is to be cultured in the bio reactor is preferred, the biological material normally being at least partially liquid.

The illustrated method of providing electrical power to the light emitting diodes 11 is through the provision of power supply support “legs” 14. Typically, each leg 14 is a positive or negative terminal connected in turn to the power source. The support legs 15 extend perpendicularly to the plane of the support 12.

As illustrated, an inner terminus 16 of the support legs 15 (being on the side of the support 12 with the LED's 11) stand slightly proud of an upper surface of the support 12. The respective inner termini 16 can be used as positive location means for a cover or the optical device 13.

According to the embodiment illustrated, there are four legs provided, three positive terminal legs 15A, 15B and 15C and a single negative terminal leg 15D. All of the terminal legs 15 have appropriate electrical wiring connections to the light emitting diodes 11.

The support of the illustrated embodiment is provided as a tiered system with an outer plate 17 and a spaced inner plate 18. As illustrated, the outer plate 17 is of larger dimension than the inner plate 18. In the illustrated embodiment, the inner plate 18 is maintained above the outer plate 17 by a shoulder wall 19 which is continuous in order to define an internal space 20 between an inner surface of the outer plate 17, the outer surface of the inner plate 18 and the inner surface of the shoulder wall 19.

As illustrated, the light emitting diodes 11 are mounted on an appropriate mounting plate 21 which is itself then subsequently mounted on the inner plate 18 of the support 12. Preferably, the electrical wiring connections 14 extend through the support 12 to connect with an under side of the mounting plate 21 as illustrated particularly in FIG. 5.

The mounting plate will normally be a printed circuit board containing electrical connections for the light emitting diodes. The mounting plate 21 is substantially centrally mounted relative to the support 12 and maintains the light emitting diodes 11 facing inwardly toward the bioreactor.

Normally, the inner plate 18 is smaller diameter than the outer plate 17. The provision of the larger dimension outer plate 17 and the shoulder wall 19 provides a section of the support 12 which can be used to positively locate the support 12 with in the bioreactor. For example, normally a terminal end of the outer expandable growth containment portion can be located about the shoulder wall 19 and inner plate 18 and abutting the outer plate .17. A circumferential clamp means may then be used to clamp or otherwise secure the outer expandable growth containment portion relative to the shoulder wall.

The apparatus and system of the invention includes a plurality of light emitting diodes (LEDs). Any number of LEDs may be provided but, a square number (9 in the illustrated embodiment) is preferred for ease of electrical connection.

As stated above, the number and combination of colours of the LEDs provided will be selected to provide an optimum wavelength of emitted light. Normally, the optimum wavelength will be within a particular range of wavelengths and the range will be chosen according to the biological material or plant material to be cultured.

The cluster of LEDs are also adjustable in order to adjust the available applied wavelengths of light during the various growth phases of the biological medium as required to stimulate or encourage growth during that particular growth phase.

Normally, the component LEDs used in the cluster for a particular application are chosen in order to provide a range of wavelengths and normally, the individual LEDs will be actuable to allow adjustment of the wavelengths emitted from a particular cluster. For example, an LED cluster of the illustrated may include LEDs of each of the component colours, for example, two red (λmax=625 nm), green (λmax=525 nm), two blue (λmax=470 nm), blue-purple (λmax=410 nm), purple (λmax=380 nm), ultraviolet ((λmax=less than 380 nm) and infrared (λmax=greater than 625 nm). By having particular LEDs in the cluster in the light and others not emit light, a wavelength range of any desired wavelength can be emitted from the apparatus. Not only that, but by switching the particular combinations which are lit and unlit, the wavelength range can be adjusted during the growth phases of the material.

The apparatus of the present invention also includes an optical device to transmit the emitted light. In its simplest form applied to use in the bioreactor field, the optical device can be a transparent or translucent cover (as is illustrated) placed over the LED cluster in order to emit the light directly into the bioreactor.

Other more advanced optical devices may be used for particular applications. For example, the optical device may be a lens which is adapted to focus or disperse the emitted light. The optical device may therefore be a convex or concave shape depending upon requirements.

Alternatively, the light emitted by the LED cluster may be conveyed, transmitted or spread using a fibre-optic cable. In order to be used in this manner, the emitted light may need to be focused to a smaller cross-section beam, and in this instance, a focusing lens may be used.

In the present specification and claims (if any), the word “comprising” and its derivatives including “comprises” and “comprise” include each of the stated integers but does not exclude the inclusion of one or more further integers.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more combinations.

1. A lighting apparatus including a plurality of coloured light emitting diodes mounted on a support, the light emitting diodes chosen to provide predetermined spectral wavelengths and an optical device to transmit the emitted light.

2. A lighting apparatus as claimed in claim 1 including a support relative to which the light emitting diodes are mounted in order to direct emitted light in one general direction.

3. A lighting apparatus as claimed in claim 2 wherein the light emitting diodes are electrically connected to a power source with electrical connections extending at least into the support from one side thereof.

4. A lighting apparatus as claimed in claim 3 wherein electrical power is provided to the light emitting diodes utilizing one or more power supply support legs, each leg being a positive or negative terminal connected in turn to the power source.

5. A lighting apparatus as claimed in claim 4 wherein a terminus of the power supply support legs stands proud of an upper surface of the support to be used as positive location means for the optical device.

6. A lighting apparatus as claimed in claim 2 wherein the support includes an outer plate and a spaced inner plate, the outer plate of larger dimension than the inner plate.

7. A lighting apparatus as claimed in claim 6 wherein the inner plate is spaced from the outer plate by an annular shoulder wall which defines an internal space between an inner surface of the outer plate, an outer surface of the inner plate and an inner surface of the shoulder wall.

8. A lighting apparatus as claimed in claim 7 wherein an electrical connection system to supply electricity to the light emitting diodes is located in the internal space.

9. A lighting apparatus as claimed in claim 2 wherein the light emitting diodes are mounted on a printed circuit board containing electrical connections for the light emitting diodes which is then subsequently mounted on the support.

10. A lighting apparatus as claimed in claim 1 wherein the number and combination of colours of the light emitting diodes provided is selected to provide an optimum wavelength of emitted light.

11. A lighting apparatus as claimed in claim 1 wherein at least some of the light emitting diodes are adjustable in order to adjust the available applied wavelengths of light emitted.

12. A lighting apparatus as claimed in claim 1 wherein the individual light emitting diodes are actuable on and off to allow adjustment of the wavelengths emitted from a particular cluster.

13. A lighting apparatus as claimed in claim 1 wherein the optical device is a transparent or translucent cover placed over the light emitting diodes in order to emit the light directly.

14. A lighting apparatus as claimed in claim 1 wherein the optical device is a lens which is adapted to focus or disperse the emitted light.

15. A bioprocess lighting system for providing light to a biological material to enhance growth, the lighting system including a plurality of light emitting diodes mounted on a support, the light emitting diodes chosen to provide optimum spectral wavelengths to the biological material and an optical device to transmit the emitted light for illumination of the biological material.

16. A bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands, the light source including a plurality of light emitting diodes mounted on a support, the light emitting diodes chosen to provide optimum spectral wavelengths to the culture medium and an optical device to transmit the emitted light for illumination of the culture medium.

17. A bioreactor including a rigid core associated with a light source, an outer expandable growth containment portion located concentrically about the rigid core, a lower end portion associated with an inlet to supply culture medium and an outlet, and an upper end cap with an opening therein through which the rigid core passes, wherein the outer containment portion expands as the cellular biological material contained in said portion grows and expands radially under pressure exerted by the growth of the biological material.
